(19) United States
(12) Patent Application Publication
(10) Pub. No.: US 2010/0025222 A1
(43) Pub. Date: Feb. 4, 2010

(54) METHOD OF FORMING PORE IN GRAPHITIC-CARBON NANOMATERIAL AND METHOD OF INTRODUCING OXYGEN-CONTAINING GROUP INTO PORE

(75) Inventors: Sumio Iijima, Minato-ku (JP); Masako Yudasaka, Minato-ku (JP); Minfang Zhang, Tsukuba-shi (JP)

Correspondence Address: SUGHRUE MION, PLLC, 2100 PENNSYLVANIA AVENUE, N.W., SUITE 800, WASHINGTON, DC, 20037, US

(73) Assignee: NEC CORPORATION, Minato-ku, Tokyo (JP)

(21) Appl. No.: 12/514,727

(22) PCT Filed: Nov. 16, 2007
(86) PCT No.: PCT/JP2007/072326

§ 371 Date: Aug. 4, 2009

(30) Foreign Application Priority Data
Nov. 17, 2006 (JP) 2006-312272

(51) Int. Cl. B29C 44/00; C07C 27/20

(52) U.S. Cl. 204/157.6; 264/414

(57) ABSTRACT

Provided are a method of forming pores in a graphitic carbon nanomaterial and a method of introducing an oxygen-containing group into the pores, in which the rate of pore formation in the wall of a graphitic carbon nanomaterial can be heightened and the amount of the oxygen-containing group, especially the carboxyl group to be introduced can be significantly increased. The method of forming pores in a graphitic carbon nanomaterial of the invention is characterized by forming pores in the wall of a graphitic carbon nanomaterial in the presence of an oxidizing agent while the nanomaterial is irradiated with a light from a light source including a light having a wavelength at which the oxidizing agent is activated.

### TECHNICAL FIELD

The present invention relates to a method of forming pores in a graphitic carbon nanomaterial and to a method of introducing an oxygen-containing group into the pores.

### BACKGROUND ART

A graphitic carbon nanomaterial such as carbon nanotubes and carbon nanohorns is composed of a graphite sheet of which the major part of the structure is a regular 6-membered ring configuration structure; and as a material having specific electric properties and having chemically, mechanically and thermally stable properties, it is actively studied and developed from various standpoints not only in an energy field but also in other various fields information communication, aviation/space, biotechnology/medical care, etc.

The inventors of this application have, after discovery of carbon nanotubes, studied oxidation of samples containing carbon nanotubes, and have discovered that the nanotubes start to react first at their tip parts to form pores therein (Non-Patent Document 1). Further, the inventors have previously proposed methods of forming pores in the wall of a graphitic carbon nanomaterial such as carbon nanotube or carbon nanohorn aggregates (Patent References 1 to 4).

The method of Patent Reference 1 comprises keeping a single-layer carbon nanotube in a dry reactive gas at a temperature falling within a range of from 200 to 600° C. for at least 1 minute thereby not only to remove the end cap of the single-layered carbon nanotube but also to form pores having a diameter of from 1 to 2 nm in the tube wall.

The method of Patent Reference 2 comprises oxidizing a single-layer carbon nanohorn aggregate in air to form pores in the horn wall.

The method of Patent Reference 3 comprises irradiating a graphitic carbon nanomaterial dispersed in a liquid medium with ultrasonic waves to form pores in the wall thereof.

The method of Patent Reference 4 comprises heating a graphitic carbon nanomaterial in a vapor current containing steam and/or carbon dioxide and an inert gas to thereby form pores in the wall while controlling their size in a simplified manner with no damage of contamination, defect formation or combustion given thereto.

### Patent Reference 1: JP-A 2002-097008

### Patent Reference 2: JP-A 2002-326032

### Patent Reference 3: JP-A 2003-205499

### Patent Reference 4: JP-A 2006-188393

### Non-Patent Reference 1: Nature, Vol. 361, No. 6410, pp. 333-334 (1993)

### DISCLOSURE OF THE INVENTION

### Problems that the Invention is to Solve

However, in the above-mentioned pore formation methods, the rate of pore formation is low, and for example, when pores are formed in the walls of single-layer carbon nanotubes according to the methods, it may take a long time of, for example, 1 week or so depending on conditions.

It is known that an oxygen-containing group such as a carboxyl group, a carbonyl group, a phenol group, a lactone group or the like may be introduced into the pore edges of the graphitic carbon nanomaterial with pores formed therein; however, in the above-mentioned pore formation methods, the amount of the functional group to be introduced into the pore edges is not so large and the amount could not be controlled. In particular, the method of pore formation through oxidation at a high temperature is defective in that the type of the functional group is limited.

The present invention has been made in consideration of the above-mentioned situation, and its object is to provide a method of forming pores in a graphitic carbon nanomaterial and a method of introducing an oxygen-containing group into the pores capable of solving the prior-art problems, capable of heightening the rate of pore formation in the wall of a graphitic carbon nanomaterial, and capable of significantly increasing the amount of an oxygen-containing group, especially a carboxyl group to be introduced.

### Means for Solving the Problems

The invention is to solve the above-mentioned problems and is characterized by the following:

1. A method of forming pores in a graphitic carbon nanomaterial, wherein pores are formed in the wall of a graphitic carbon nanomaterial in the presence of an oxidizing agent while the nanomaterial is irradiated with a light from a light source including a light having a wavelength at which the oxidizing agent is activated.

2. The method of forming pores in a graphitic carbon nanomaterial of the above 1, wherein the oxidizing agent is activated through contact with the graphitic carbon nanomaterial having absorbed the light from the light source.

3. The method of forming pores in a graphitic carbon nanomaterial of the above 1 or 2, wherein the wavelength of the light to activate the oxidizing agent falls within a range of from 250 to 500 nm.

4. The method of forming pores in a graphitic carbon nanomaterial of the any of the above 1 to 3, wherein the oxidizing agent is hydrogen peroxide, oxygen gas, carbon monoxide gas or carbon dioxide gas.

5. The method of forming pores in a graphitic carbon nanomaterial of the above 4, wherein the oxidizing agent is hydrogen peroxide.

6. The method of forming pores in a graphitic carbon nanomaterial of the any of the above 1 to 5, wherein the graphitic carbon nanomaterial is a carbon nanotube or a carbon nanohorn.

7. A method of introducing an oxygen-containing group into the pores of a graphitic carbon nanomaterial, wherein pores are formed in the wall of a graphitic carbon nanomaterial in the presence of hydrogen peroxide while the nanomaterial is irradiated with a light from a light source including a light having a wavelength at which hydrogen peroxide is activated, and an oxygen-containing group is introduced into the pore edges.

8. The method of forming pores in a graphitic carbon nanomaterial of the above 7, wherein hydrogen peroxide is activated through contact with the graphitic carbon nanomaterial having absorbed the light from the light source.

9. The method of forming pores in a graphitic carbon nanomaterial of the above 7 or 8, wherein the wavelength of the light to activate hydrogen peroxide falls within a range of from 250 to 500 nm.

10. The method of introducing an oxygen-containing group into the pores of a graphitic carbon nanomaterial of any of the above 7 to 9, wherein the oxygen-containing group includes at least a carboxyl group.

11. The method of introducing an oxygen-containing group into the pores of a graphitic carbon nanomaterial of any of the above 7 to 10, wherein the graphitic carbon nanomaterial is a carbon nanotube or a carbon nanohorn.

### EFFECT OF THE INVENTION

According to the pore formation method of the invention, pores are formed in the wall of a graphitic carbon nanomaterial while the nanomaterial is irradiated with a light from a light source including a light having a wavelength at which an oxidizing agent is activated, and therefore the rate of pore formation in the wall of the graphitic carbon nanomaterial can be heightened, and, for example, the pores can be formed at a rate higher by at least two times than that in conventional methods.

Further, according to the pore formation method and the oxygen-containing group introduction method of the invention, hydrogen peroxide is used as an oxidizing agent, and the amount of the oxygen-containing group to be introduced, especially the amount of a carboxyl group to be introduced can be significantly increased.

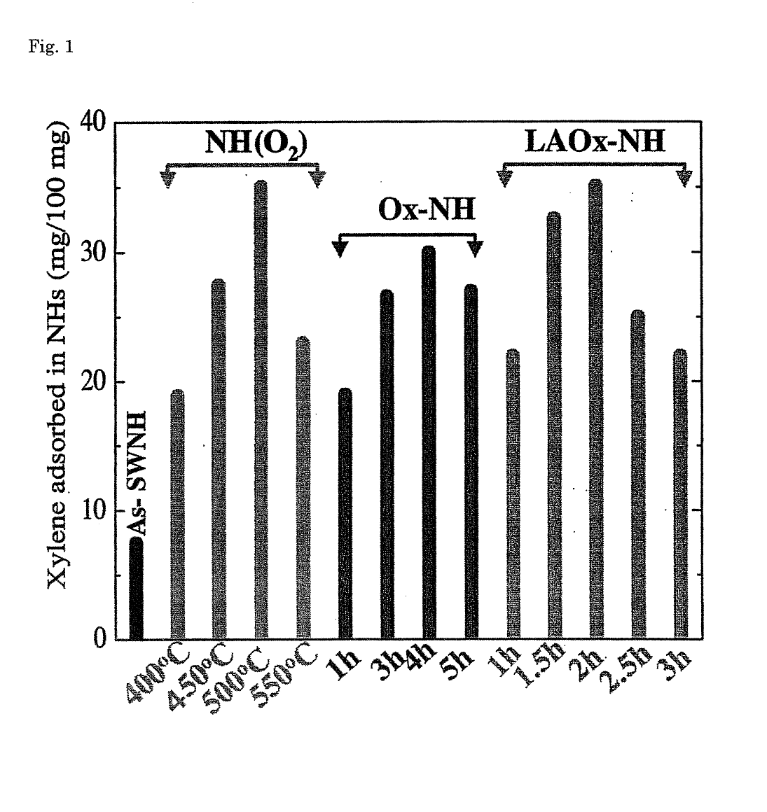

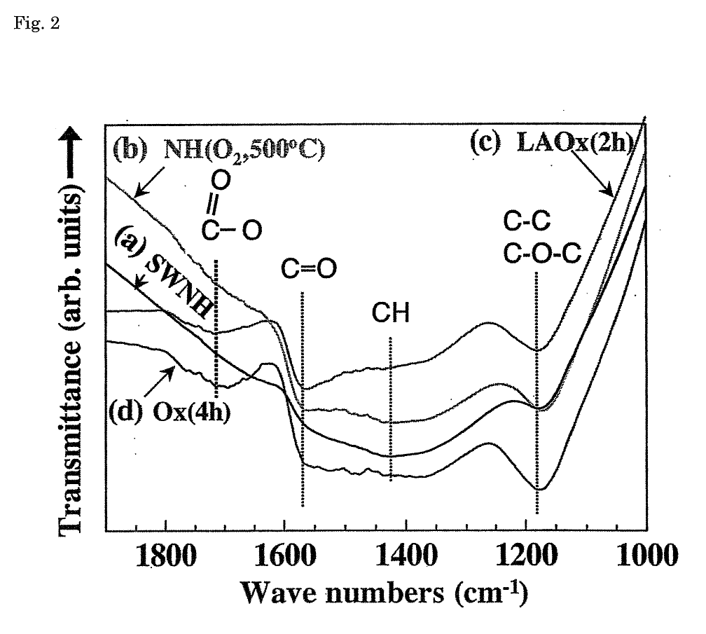

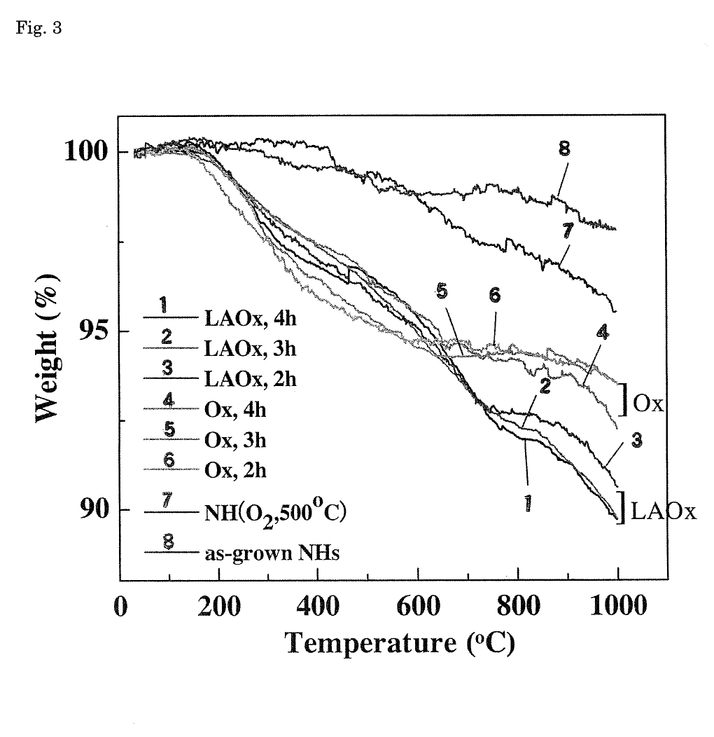

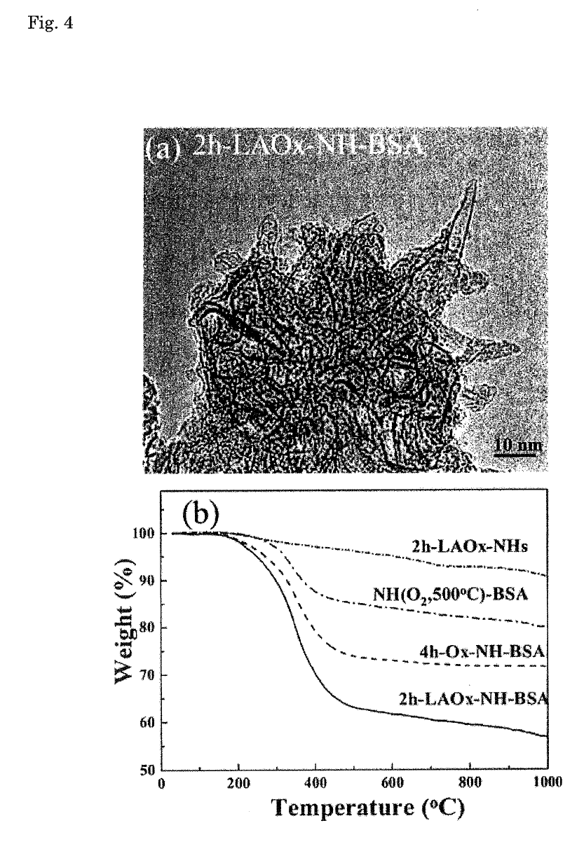

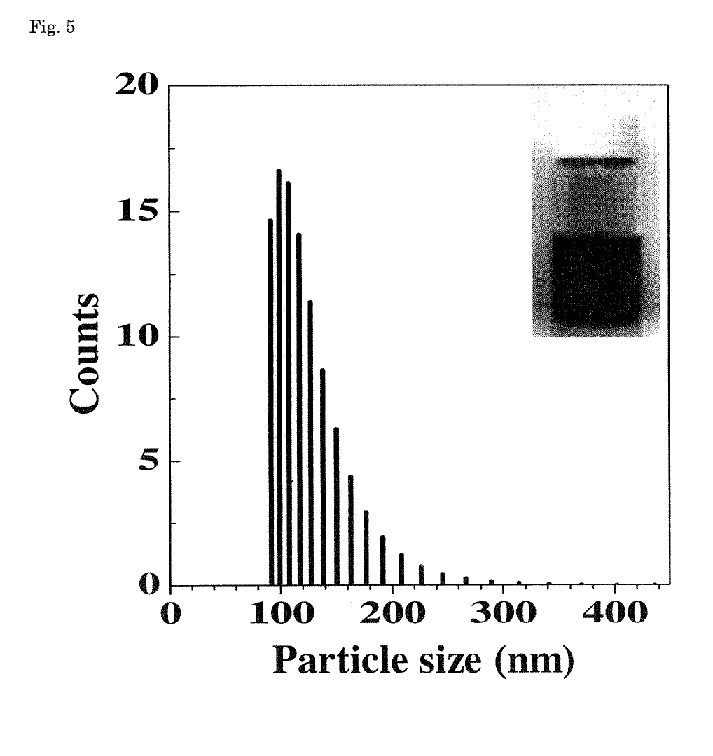

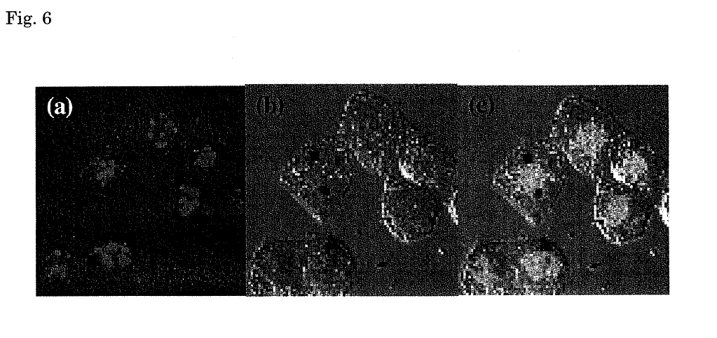

### BRIEF DESCRIPTION OF THE DRAWINGS

[FIG. 1] It is a graph showing the amount of xylene adsorbed at room temperature by pores-formed carbon nanohorn aggregates.

[FIG. 2] It shows IR absorption spectra of pores-formed carbon nanohorn aggregates.

[FIG. 3] It is a graph showing the results of thermal gravimetric analysis (TGA) of pores-formed carbon nanohorn aggregates.

[FIG. 4] It shows (a) a transmission electromicroscopic image of a carbon nanohorn aggregate reacted with BSA, and (b) the results of thermal gravimetric analysis (TGA) thereof.

[FIG. 5] It is a graph showing the particle size distribution of a carbon nanohorn aggregate reacted with BSA.

[FIG. 6] It shows pictures of (a) LAOx-NH(2h)-BSA, (b) human lung cancer cells H460, and (c) LAOx-NH(2h)-BSA taken in H460 cells.

### BEST MODE FOR CARRYING OUT THE INVENTION

The invention is characterized by the above, and its embodiments are described below.

As the graphitic carbon nanomaterial to be processed for forming pores therein in the invention, there may be mentioned a substance that contains a graphite sheet having a 6-membered ring configuration structure as the main structure thereof, and its concrete examples include carbon nanotubes, carbon nanohorns, graphite nanofibers, carbon nanocones, fullerenes, nanocapsules, etc.

Carbon nanotubes include so-called single-layer carbon nanotubes where the graphite sheet to form the tube is a single layer, and multilayer carbon nanotubes where a large number of graphite sheet cylinders are piled up like nest boxes; and any of these may be used in the invention. Carbon nanotubes having, for example, an outer diameter of at most 1 μm and an inner diameter of at least 0.4 nm may be used; and they may be individually separated from each other, or a large number of such carbon nanotubes may be bundled up bundles.

Carbon nanohorns have a horned structure seeming to be one graphite sheet rolled up like a hollow cone; and not having a constant tube diameter like that of carbon nanotubes, they are so constituted that their diameter gradually and continuously increases starting from the closed tip end as the top, and include various heterogeneous structures where the wall is folded or not.

Carbon nanohorns may have a morphology of carbon nanohorn aggregates of spherical particles where a large number of carbon nanohorns are aggregated with their conical closed tip ends aligning from the center toward the outside.

Carbon nanocones have a structure of one graphite sheet rolled up like a hollow cone, in which the conical angle may vary differently.

The graphitic carbon nanomaterial to be processed for forming pores therein in the invention may contain any other element than carbon, such as B and N, and may have an inclusion morphology in any other substance.

Specific examples of the oxidizing agent for use in the invention include hydrogen peroxide, oxygen gas, carbon monoxide gas, carbon dioxide gas, etc. The oxidizing agent may be activated and decomposed through energy transfer or electron transfer from a graphitic carbon nanomaterial having absorbed the light from a light source falling within a region of UV to visible light, and the decomposed component promotes the oxidative pore formation in the graphitic carbon nanomaterial.

For example, when hydrogen peroxide is used as the oxidizing agent, a radical having extremely high reactivity such as HOO., HO. or the like is generated according to the above-mentioned mechanism of irradiation with light from a light source. The reactive radical reacts with the defects and the like in the wall (including the tip) of a graphitic carbon nanomaterial and forms pores in the wall while releasing O₂ and CO through decomposition.

In case where hydrogen peroxide is used, pores are formed in the wall and simultaneously a large quantity of an oxygen-containing group is introduced into the pore edges. In particular, the oxygen-containing group includes many carboxyl groups.

On the other hand, in case where pores are formed with no irradiation with light like conventionally, it may be considered that reaction may occur mainly between the graphitic carbon nanomaterial and an ion such as HOO⁻; however, the activity of radical is far stronger than the activity of ion, and therefore in the invention, the reaction speed, or that is the rate of pore formation can be significantly heightened.

In case where oxygen gas is used as the oxidizing agent, the rate of pore formation may be heightened through activation of oxygen molecules as a result of irradiation with light; however, as shown in Examples to be given hereinafter, the amount of the oxygen-containing group, especially the amount of the carboxyl group to be introduced is low as compared with that in the case where hydrogen peroxide is used as the oxidizing agent.

In case where an oxidizing reagent such as hydrogen peroxide is used as the oxidizing agent, the oxidative pore formation treatment may be attained through contact of a graphitic carbon nanomaterial with the oxidizing agent with irradiation thereof with light, for example, in a liquid medium at −20 to 200° C.

In case where an oxidizing gas such as oxygen gas, carbon monoxide gas, carbon dioxide gas or the like is used as the oxidizing agent, the oxidative pore formation treatment may be attained through contact of a graphitic carbon nanomaterial with the oxidizing gas with irradiation thereof with light, under a suitably controlled pressure condition within a temperature range of, for example, from 200 to 600° C. for oxygen gas, and from 500 to 1200° C. for carbon monoxide gas or carbon dioxide gas.

Irradiation with light is attained, using a light source that includes a light having a wavelength at which the oxidizing agent is activated. The wavelength of light at which the oxidizing agent is activated is within a range of UV to visible light as the light absorption range of a graphitic carbon nanomaterial, preferably within a range of from 250 to 500 nm, since the oxidizing agent is activated through energy transfer or electron transfer from the graphitic carbon nanomaterial having absorbed light as so mentioned in the above.

Specific examples of the light source that includes a light having such a wavelength range include mercury lamp, xenon lamp, laser, etc.; however, various light sources including white light sources and monochromatic light sources can be used with no specific limitation so far as they are satisfactory in point of the light intensity and the irradiation dose within the wavelength range.

Through the irradiation with light, pores may be formed in the wall of a graphitic carbon nanomaterial at a rate higher, for example, by at least 2 times than that in the case with no irradiation with light; and depending on conditions, desired pores may be formed therein within an irradiation time of from 1 millisecond to 3 days or so.

The irradiation with light may be attained entirely during the oxidation treatment or may be attained only partly within a desired period of time during the oxidation treatment.

In case where hydrogen peroxide is used as described in the above, a large quantity of an oxygen-containing group such as a carboxyl group may be introduced into the pore edges of the graphitic carbon nanomaterial through irradiation with light. Further, controlling the condition in irradiation with light may bring about a possibility of imparting various functions to the processed graphitic carbon nanomaterial.

The invention is described in more detail with reference to the following Examples. Needless-to-say, the invention is not limited by the following exemplification.

### EXAMPLES

A carbon nanohorn aggregate (SWNH) was used as a graphitic carbon nanomaterial, and pores were formed therein according to the following three methods, and these were compared with each other.

(1) In oxygen gas, the material is heated at a predetermined temperature for 15 minutes (NH(O₂)).

(2) In an aqueous hydrogen peroxide solution, the material is heated at 100° C. for a predetermined period of time (Ox-NH).

(3) In an aqueous hydrogen peroxide solution, the material is heated at 100° C. for a predetermined period of time while irradiated with light (LAOx-NH).

Regarding the condition in irradiation with light, the light source was a xenon lamp (250 W), the light intensity was up to 3 W, and the irradiation time was from 1 to 5 hours.

### <A> Xylene Adsorption:

The pores-formed carbon nanohorn aggregates processed according to the treatment were analyzed for xylene adsorption at room temperature. The results are shown in FIG. 1. From FIG. 1, it is known that the xylene adsorption by the carbon nanohorn aggregate heated in oxygen gas at 500° C. for 15 minutes according to the above method (1) [NH(O₂, 500° C.)] and that by the carbon nanohorn aggregate heated in an aqueous hydrogen peroxide solution at 100° C. for 2 hours while irradiated with light according to the above method (3) [LAOx-NH(2h)] were the largest. It is known that, as compared with that of the unprocessed carbon nanohorn aggregate, the inner capacity of these carbon nanohorn aggregates is the largest. It is further known that the pores on the same level were formed at a rate higher by at least 2 times through irradiation with light.

### <B> Introduction of Substituent:

The carbon nanohorn aggregates having the largest inner capacity of those processed for pore formation according to the above three methods were analyzed for IR absorptiometry, and the results are shown in FIG. 2. As a result, [Ox-NH(4h)] according to the method (2) and [LAOx-NH(2h)] according to the method (3) gave peaks (1585 cm⁻¹ (C═O), 1725 cm⁻¹ (—COO)) peculiar to carboxyl group. The unprocessed carbon nanohorn aggregate and [NH(O₂, 500° C.)] according to the method (1) did not give the obvious peaks of carboxyl group. These confirm that the pore formation in the carbon nanohorn aggregate according to the method (2) and the method (3) brings about introduction of a functional group such as a carboxyl group into the pore edges.

For estimating the amount of the carboxyl group, the samples were processed for thermal gravimetric analysis (TGA) in He. The results are shown in FIG. 3. In addition, the samples were analyzed through mass spectrometry, which confirm that the amount of the carboxyl group and other oxygen-containing groups is the largest in the case of the method (3) of the invention and that the amount of the substituent such as the carboxyl group in the part of the pores formed according to the pore formation method combined with irradiation with light increases.

### <C> Chemical Modification:

BSA (bovine serum albumin), a type of protein was reacted with the oxygen-containing group such as the carboxyl group introduced into the above carbon nanohorn aggregates. BSA particles (2 to 3 nm) or their ranged aggregates adhering to the carbon nanohorn aggregates were confirmed through observation with a transmission electronic microscope (TEM), and the result is shown in FIG. 4(a). The amount of the adhering BSA was estimated from the weight loss in TGA in He, and the results are shown in FIG. 4(b). The results confirm that the amount of BSA having adhered to the carbon nanohorn aggregates processed for pore formation therein according to the above method (3) is the largest. The results well correspond to the results indicating the largest amount of the carboxyl group in irradiation with light (FIG. 3).

In the carbon nanohorn aggregates processed for pore formation therein in the method (3) and processed for BSA adhesion thereto (LAOx-NH(2h)-BSA), BSA is hydrophilic; and therefore, as shown in FIG. 5, the aggregate was uniformly dispersed in PBS (phosphate buffered saline). The dispersion liquid was analyzed according to a light-scattering method, and the particle size of the nanohorn aggregate thus measured was larger in some degree than the particle size (80 to 100 nm) of the nude nanohorn aggregate. This reflects the fact that the increase in the particle size is by adhesion of BSA or its multimer to the nanohorn aggregate but that the BSA-adhering nanohorn aggregates disperse separately not almost associating together.

LAOx-NH(2h)-BSA well dispersed in PBS was checked for its cell affinity with human lung cancer cells H460, and it has been found that LAOx-NH(2h)-BSA were taken in the H460 cells. In FIG. 6, (a) shows LAOx-NH(2h)-BSA, (b) shows human lung cancer cells H460, and (c) shows LAOx-NH(2h)-BSA taken in H460 cells. The size of the carbon nanohorn aggregate is from 80 to 100 nm or so, and it is expected that the aggregate may be specifically accumulated in a cancer tissue (passive targeting effect). Further, with BSA or the like added thereto so as to be taken into individual cancer cells, the carbon nanohorn aggregate may be expected to have an increased effect as a drug carrier.

1. A method of forming pores in a graphitic carbon nanomaterial, wherein pores are formed in the wall of a graphitic carbon nanomaterial in the presence of an oxidizing agent while the nanomaterial is irradiated with a light from a light source including a light having a wavelength at which the oxidizing agent is activated.

2. The method of forming pores in a graphitic carbon nanomaterial as claimed in claim 1, wherein the oxidizing agent is activated through contact with the graphitic carbon nanomaterial having absorbed the light from the light source.

3. The method of forming pores in a graphitic carbon nanomaterial as claimed in claim 1, wherein the wavelength of the light to activate the oxidizing agent falls within a range of from 250 to 500 nm.

4. The method of forming pores in a graphitic carbon nanomaterial as claimed in claim 1, wherein the oxidizing agent is hydrogen peroxide, oxygen gas, carbon monoxide gas or carbon dioxide gas.

5. The method of forming pores in a graphitic carbon nanomaterial as claimed in claim 4, wherein the oxidizing agent is hydrogen peroxide.

6. The method of forming pores in a graphitic carbon nanomaterial as claimed in claim 1, wherein the graphitic carbon nanomaterial is a carbon nanotube or a carbon nanohorn.

7. A method of introducing an oxygen-containing group into the pores of a graphitic carbon nanomaterial, wherein pores are formed in the wall of a graphitic carbon nanomaterial in the presence of hydrogen peroxide while the nanomaterial is irradiated with a light from a light source including a light having a wavelength at which hydrogen peroxide is activated, and an oxygen-containing group is introduced into the pore edges.

8. The method of forming pores in a graphitic carbon nanomaterial as claimed in claim 7, wherein hydrogen peroxide is activated through contact with the graphitic carbon nanomaterial having absorbed the light from the light source.

9. The method of forming pores in a graphitic carbon nanomaterial as claimed in claim 7, wherein the wavelength of the light to activate hydrogen peroxide falls within a range of from 250 to 500 nm.

10. The method of introducing an oxygen-containing group into the pores of a graphitic carbon nanomaterial as claimed in claim 7, wherein the oxygen-containing group includes at least a carboxyl group.

11. The method of introducing an oxygen-containing group into the pores of a graphitic carbon nanomaterial as claimed in claim 7, wherein the graphitic carbon nanomaterial is a carbon nanotube or a carbon nanohorn.
